(19) United States
(12) Patent Application Publication
(10) Pub. No.: US 2019/0112678 A1
(43) Pub. Date: Apr. 18, 2019

(54) COMPOSITION AND METHOD FOR ENHANCING CHITIN-CONTAINING FERTILIZERS

(71) Applicant: Osprey Biotechnics, Inc., Sarasota, FL (US)

(72) Inventors: Christopher J. Reuter, Parrish, FL (US); Steven J. MacKenzie, Sarasota, FL (US)

(73) Assignee: Osprey Biotechnics, Inc., Sarasota, FL (US)

(21) Appl. No.: 15/787,109

(22) Filed: Oct. 18, 2017

(51) Int. Cl. C12R 1/10; C05F 11/08; C12N 1/20; C05C 11/00; C05F 1/00; C08B 37/08; C05G 3/00

(52) U.S. Cl. CPC C12Y 302/01014; C12R 1/10; C05F 11/08; C05G 3/007; C05C 11/00; C05F 1/007; C08B 37/003; C12N 1/20

(57) ABSTRACT

A composition and process for improving the growth-promoting effectiveness of chitin-based fertilizers involves contacting the chitin-based material with a bacteria that secretes chitinase.

### CROSS-REFERENCE TO RELATED APPLICATIONS

Not Applicable.

### FIELD OF THE DISCLOSURE

This disclosure relates to compositions and processes for promoting plant growth, and more particularly to improved chitin-based fertilizers and processes for improving the effectiveness of chitin-based fertilizers.

### BACKGROUND OF THE DISCLOSURE

Chitin-based fertilizers have been shown to improve plant growth, with significant improvements being reported for field crops such as daikon radishes, cabbage, soybean sprouts, sweet basil, grapevine, and for ornamental plants such as Gerbera and Dendrobiuin orchids. However, there are also reports that application of chitin-based fertilizers did not significantly improve growth, biomass production, or yield for rice, soybean and maize.

### SUMMARY OF THE DISCLOSURE

The disclosed compositions and processes are directed to improving the effectiveness of chitin-based fertilizers by combining them with strains of bacteria that secrete chitinase.

The compositions include at least one strain of bacteria that secretes chitinase intimately combined with at least one chitin-based material, such that upon application to a plant, seed from which a plant is grown, or to a place where a plant is grown, the bacteria secretes sufficient chitinase to accelerate hydrolysis of glycosidic bonds in the chitin-based material. This makes nitrogen and phosphorous contained in the chitin more readily available to a growing plant, resulting in significantly improved growth rates, biomass production rates, and/or higher crop yields.

The processes of this disclosure involve applying a combination of a chitin-based material and a chitinase-secreting bacteria to a plant, plant seed, or soil or other environment in which a plant is grown to enhance the ability of the chitin-based material to promote plant growth and improve crop biomass production and yields.

### DETAILED DESCRIPTION OF THE DISCLOSED EMBODIMENTS

Two strains of bacteria were selected that demonstrated the ability to secrete chitinase and evaluated to determine if one or both of the strains improves the performance of chitin-containing fertilizers. The strains selected for evaluation were Bacillus licheniformis (OBT 618) and a strain of Paenibacillus ehimensis (PE). The strains were combined with the chitinous substrates crab shell and shrimp shell. The chitinous substrate and bacteria can be applied to the soil as a dry product where the bacteria are adhered to the chitin substrate with a binder. Examples of suitable binders include polyvinyl alcohol, starches, carboxymethyl cellulose, dextrin, methyl cellulose and hydroxymethyl cellulose. Alternatively, the chitinous substrate can be combined with the bacteria in a liquid and incubated for several days and the liquid applied to the soil. The strains were selected for their ability to produce chitinase, an enzyme that hydrolyzes glycosidic bonds in the chitin polymer. Chitin is a long chain polymer of N-acetylglucosamine and is found in the exoskeleton of insects and crustaceans and in the cell wall of fungi. Nitrogen from chitin contributes to the high nitrogen content found in ground crab shell and shrimp shell. Shell meal from these sources contain nitrogen in the range of 4% to 6%, and are valuable organic fertilizers. In addition to containing nitrogen, these meals contain phosphorous in the range of 3% to 6% which enhances their nutritional value.

Chitin is an insoluble material and conversion of chitin to soluble monomers or dimers of N-acetyl-D-glucosamine (GlcNAc) by chitinase is the first step in the degradation (fermentation) of this product. After solubilization of the GlcNAc subunits, carbon and nitrogen comprising the GlcNAc subunits can be mineralized, mineralized and incorporated into plant or microbial biomass or incorporated into microbial biomass directly. Mineralization of nitrogen from chitinous fertilizers must occur for them to be effective in nitrogen depleted soils. Experiments were designed to determine whether combining one of the chitin producing strains B. licheniformis (OBT 618) and P. ehimensis (PE) with a chitinous substrate will stimulate the fertilizer efficacy of the chitinous substrate by enhancing the rate of nitrogen mineralization from chitin when applied to the soil. It is also proposed to combine B. licheniformis (OBT 618) or P. ehimenis (PE) with a chitinous substrate in a liquid medium and grow the bacterium on the substrate prior to application to a plant or soil. This allows hydrolysis of the chitin substrate prior to application and allows fertilizer derived from shells or other chitin-based materials to be applied as a liquid. This has the advantage of allowing growers to apply chitinous fertilizers through their irrigation systems. One of the strains, P. ehimensis (PE), produces a diffusible compound that is antagonistic to the fungus Fusarium oxysporum. Combining this strain with chitin could enhance the production of the diffusible compound in soil contributing to a repressive effect of the strain.

### Strain Descriptions

B. Licheniformis (OBT 618) was obtained from the ARS culture collection from a population study looking at Bacillus species in the Sonoran Desert. The original strain designation was NRRL-B-23318. It was subsequently found to produce a large clearance zone when grown on colloidal chitin medium, indicating it secreted a high level of chitinase. It was not found to secrete any substances that inhibited the growth of fungi.

P. ehimensis (PE) was also obtained from the ARS culture collection, where it is designated strain NRRL-B-23118. It is the type specimen for this species. It was originally isolated from soil in Japan in a screen for chitinase producing organisms and described as Bacillus ehimensis. It was later assigned to the Paenibacillus genus. A colloidal chitin clearing assay was used to confirm that the strain produces chitinase. We also looked at the strain's ability to secrete anti-fungal compounds. It was shown to produce a highly antagonistic compound active against a Fusarium strain.

### EXPERIMENTATION

Solutions of crab meal (4-3-0) purchased from Down to Earth Natural Fertilizers were prepared by suspending the crab meal in water at 3.5% and then sterilizing the solutions in an autoclave. A flask containing 100 ml of the suspension was inoculated with a colony of B. licheniformis (OBT 618) and the flask was agitated for 5 days. The fertilizer efficacy of inoculated and non-inoculated crab meal suspensions were tested by treating basil seedlings emerging from 200 grams of nutrient deficient soil (25% water content) with the soluble portion of the crab meal solution at 34 ppm nitrogen per gram of soil (24 ml of 3.5% crab meal into 1 kg of soil). This treatment was repeated one week later. Also, soils were treated with 66 ppm potassium in the form of potassium chloride to supplement the fertilizer because it lacks this nutrient. After two weeks from the first treatment soil was washed from roots, the plants were dried with absorbent paper and each plant was weighed. Crab meal treatments were compared to a control treatment that was not fertilized and inoculated and to each other. The results are reported in Table 1. The results indicate that B. licheniformis strain OBT 618 solubilized nutrients from the crab meal suspension that were utilizable by basil seedlings.

TABLE 1
Mean fresh weights of basil plants two weeks after
emergence from soil when treated with solutions prepared
from inoculated and non-inoculated crab meal.

| | | Mean fresh |
|---|---|---|
| | | weight ± standard |
| Treatment | n | error^{x} |
| Control | 6 | 0.602 ± 0.078 |
| 3.5% Crab meal | 6 | 0.642 ± 0.042^{ns} |
| 3.5% Crab meal + B. licheniformis | 6 | 1.568 ± 0.140^{*†} |
| (OBT 618) | | |

^{x}The abbreviation ^{ns} indicates that the mean is not statistically significant from the control.
Symbols ^{*} and ^{†} indicate statistically significant from the control and corresponding non-inoculated crab meal, respectively, at P < 0.001.

Shrimp meal (6-6-0) suspensions were made at 3.5% using the same procedure as that used for crab shell (16.2 ml 3.5% shrimp meal per kg of soil). Like results with meal, the soluble portion of the shrimp meal suspension stimulated plant growth when inoculated with B. licheniformis (OBT 618) and did not stimulate growth in the absence of the inoculant (Table 2).

TABLE 2
Mean fresh weights of basil plants two weeks after
emergence from soil when treated with solutions prepared
from inoculated and non-inoculated shrimp meal.

| | | Mean fresh |
|---|---|---|
| | | weight ± standard |
| Treatment | n | error^{x} |
| Control | 4 | 0.110 ± 0.041 |
| 3.5% Shrimp meal | 4 | 0.131 ± 0.0121^{ns} |
| 3.5% Shrimp meal + B. licheniformis | 4 | 1.347 ± 0.181^{*†} |
| (OBT 618) | | |

^{x}The abbreviation ^{ns} indicates that the mean is not statistically significant from the control.
Symbols ^{*} and ^{†} indicate statistically significant from the control and corresponding non-inoculated shrimp meal, respectively, at P < 0.001.

P. ehimensis (PE) grew well on shrimp meal, but failed to grow on crab meal. The ability of this strain to promote the growth potential of shrimp meal suspensions was compared to B. licheniformis (OBT 618). Plant growth stimulated by a combination of shrimp meal+P. ehimensis (PE) was indistinguishable from growth stimulated by shrimp meal+B. licheniformis (OBT 618) (Table 3).

TABLE 3
Mean fresh weights of basil plants two weeks after
emergence from soil when treated with solutions prepared
from shrimp meal inoculated with different strains of bacteria.

| | | Mean fresh |
|---|---|---|
| | | weight ± standard |
| Treatment | n | error^{x} |
| Control | 6 | 0.738 ± 0.067 |
| 3.5% Shrimp meal + B. licheniformis | 6 | 1.905 ± 0.139^{*} |
| (OBT 618) | | |
| 3.5% Shrimp meal + P. ehimensis (PE) | 6 | 1.864 ± 0.171^{*ns} |

^{x}The symbol ^{*} indicates that the mean is statistically significant from the control at P < 0.001.
The abbreviation ^{ns} indicates that this mean is not statistically different from the mean for B. licheniformis (OBT 618) treated shrimp meal.

In two other experiments, B. licheniformis (OBT 618) and P. ehimensis (PE) spores from a dried powder were suspended in 1% carboxymethylcellulose at 1×10⁸ spores/ml. These suspensions were mixed with crab meal or shrimp meal at a ratio of 10 parts meal to 1 part suspension. After mixing, the meal was dried overnight yielding a solid at 1×10⁷ spores/g. The meals were mixed with nutrient depleted potting soil at 100 ppm nitrogen (0.24% for crab meal and 0.164% for shrimp meal) from the chitinous substrate. Soils were also supplemented with 100 ppm potassium in the form of potassium chloride. Pots were seeded and weights of plants recorded after one month. In the first experiment, looking at the effect of B. licheniformis (OBT 618) on growth of basil fertilized with crab meal, the crab meal stimulated growth without the inoculant indicating sufficient chitinolytic bacteria in the soil to effectively mineralize the crab meal. Addition of B. licheniformis (OBT 618) to the crab meal did not stimulate growth further (Table 4).

TABLE 4
Mean fresh weights of basil plants 1 month after
seeding when treated with crab meal or crab
meal and B. licheniformis (OBT 618).

| | | Mean fresh |
|---|---|---|
| | | weight ± standard |
| Treatment | n | error^{x} |
| Control | 4 | 0.600 ± 0.061 |
| Crab meal | 4 | 2.508 ± 0.059^{*} |
| Crab meal + B. licheniformis (OBT 618) | 4 | 2.534 ± 0.088^{*ns} |

^{x}The symbol ^{*} indicates that the mean is statistically significant from the control at P < 0.001.
The abbreviation ^{ns} indicates that this mean is not statistically different from the mean for crab meal alone.

Shrimp meal also stimulated growth of basil in the absence of an inoculant. However, unlike the results for crab, B. licheniformis (OBT 618) enhanced the fertilizer effect. This result was also observed for the bacterium P. ehimensis (PE) (Table 5). The degree to which the bacteria increased basil biomass was not different between strains.

TABLE 5
Mean fresh weights of basil plants 1 month after
seeding when treated with shrimp meal or
shrimp meal plus an inoculant.

| | | Mean fresh |
|---|---|---|
| | | weight ± standard |
| Treatment | n | error^{x} |
| Control | 4 | 0.600 ± 0.061 |
| Shrimp meal | 4 | 3.601 ± 0.264^{*} |
| Shrimp meal + B. licheniformis | 4 | 4.391 ± 0.099^{*†} |
| (OBT 618) | | |
| Shrimp meal + P. ehimensis (PE) | 4 | 4.521 ± 0.31^{*†ns} |

^{x}The symbol ^{*} indicates that the mean is statistically significant from the control at P < 0.001.
The symbol ^{†} indicates that the mean was statistically significant from the mean for uninoculated shrimp meal P < 0.05.
The abbreviation ^{ns} indicates that this mean is not statistically different from the mean for shrimp meal inoculated with B. licheniformis (OBT 618).

The PE strain of P. ehimensis inhibits the growth of Fusarium oxysporum f. sp. lycopersicion on TSA agar plates. We tested its efficacy for reducing the number and germination percentage of conidia from this species. Fusarium conidia at 1×10⁶/ml were treated with 0.1% glucose, 0.02% soy peptone and 0.01% yeast extract (germination medium) to stimulate germination. In two other treatments, 50% effluent from strain PE growing in TSB or 3.5% shrimp meal was included with the germination medium. Only the shrimp effluent was effective at reducing conidia germination (Table 6). However, it did not reduce spore counts to levels below that observed in a treatment where the conidia were not stimulated to germinate (Table 7).

TABLE 6
Percent germination of Fusarium conidia with or without a nutrition
source and with a nutrition source and effluent from P. ehimensis
growing in TSB or 3.5% shrimp meal

| | | % germination |
|---|---|---|
| | % germination | (Shrimp meal |
| Treatment | (TSB effluent)^{x} | effluent)^{x} |
| No germination medium | 15.4%* | 4.21%* |
| Germination medium only | 62.3% | 71.8% |
| Germination medium plus PE effluent | 86.6% | 0%* |

^{x}The symbol * indicates that the mean is statistically significant from the control for this experiment, germination medium only, at P < 0.001.

TABLE 7
Number of Fusarium conidia after treatment with or without a
nutrition source and with a nutrition source and effluent from
P. ehimensis growing in TSB or 3.5% shrimp meal

| | | Conidia/ml |
|---|---|---|
| | Conidia/ml | (Shrimp meal |
| Treatment | (TSB effluent)^{x} | effluent)^{x} |
| No germination medium | 624000 | 760000 |
| Germination medium only | 1040000 | 760000 |
| Germination medium plus PE effluent | 656000 | 640000 |

^{x}The control group for this experiment was the no germination medium. None of the two other experiments had a reduced number of conidia relative to the control.

In another experiment, Fusarium conidia were directly added to the soil at 5×10⁵ conidia/g. P. ehimensis strain PE was adhered to shrimp meal at 1×10⁸ spores/g and incorporated at 0% to 5% of the soil weight. In this experiment, Fusarium was recovered at the same rate for all soils after one week except those in which 5% shrimp meal was incorporated into the soil. Five percent shrimp meal could provide over 3000 ppm nitrogen to the soil, which would pose a hazard to the plant. For this reason, incorporating shrimp at this level isn't likely to be feasible. However, at lower concentrations of shrimp meal, bacteria might inhibit growth, but not viability of the conidia. If this is true it may still inhibit fusarium infections.

TABLE 8
Effect of shrimp meal inoculated with P. ehimensis
strain PE on recovery of Fusarium in soil

| % P. ehimensis | | Fusarium |
|---|---|---|
| inoculated | | colonies |
| shrimp meal | | recovered/g |
| in soil | Replicates | soil^{x} |
| 0% | 3 | 6.6 × 10⁵ |
| 0.2% | 3 | 7.2 × 10⁵ |
| 0.5% | 3 | 5.0 × 10⁵ |
| 1% | 3 | 5.2 × 10⁵ |
| 5% | 3 | 2 × 10³* |

^{x}Treatment labeled with * is significantly lower than 0% shrimp meal treatment.

The study results indicate that the primary benefit of the chitin degrading strains examined would be to convert solid chitin containing compounds to liquid fertilizer or to augment the fertilizer efficacy of chitin containing materials. The efficacy of shrimp meal and crab meal as fertilizers was better than other organic fertilizers we tested. This probably results from the fact that the nitrogen is bound in a carbohydrate as opposed to protein. Pre-degrading chitin from either crab shell or shrimp shell with the two selected strains of bacteria produced a good aqueous fertilizer. The B. licheniformis strain seemed to work better for this purpose as it could grow on both crab or shrimp shell and grew faster than the P. ehimensis strain. When incorporated into the soil alone (without inoculation), the shrimp meal and crab meal were good fertilizers, indicating that there are endogenous bacteria in the soil that can hydrolyze and degrade chitin. Inoculating the shrimp meal with either strain appeared to improve the fertilizer efficacy. The P. ehimensis strain was the only strain that could grow on chitin and produced anti-fungal compounds. A combination of shrimp meal and P. ehimensis did not result in the death of Fusarium conidia in the soil until the concentration of shrimp meal was 5%. The cost of this treatment and the excessively high ammonia content in the soil for this treatment might preclude it from being a viable option to control diseases caused by Fusarium species.

The described embodiments are preferred and/or illustrated, but are not limiting. Various modifications are considered within the purview and scope of the appended claims.

What is claimed is:

1. A composition comprising:
chitin and a bacteria that secretes chitinase.

2. The composition of claim 1, wherein the bacteria is Bacillus licheniformis (OBT 618).

3. The composition of claim 1, wherein the bacteria is Paenibacillus ehimensis (PE).

4. The composition of claim 1, wherein the bacteria is attached to the chitin with a binder.

5. The composition of claim 1, wherein the bacteria and chitin are present in a liquid suspension.

6. The composition of claim 1, wherein the chitin is present in the form of ground crab shell.

7. The composition of claim 1, wherein the chitin is present in the form of ground shrimp shell.

8. A composition comprising:
a soluble portion of a fermented suspension of chitin and a bacteria that secretes chitinase.

9. The composition of claim 8, wherein the bacteria is Bacillus licheniformis (OBT 618).

10. The composition of claim 8, wherein the bacteria is Paenibacillus ehimensis (PE).

11. The composition of claim 8, wherein the chitin is present in the form of ground crab shell.

12. The composition of claim 8, wherein the chitin is present in the form of ground shrimp shell.

13. A process for promoting plant growth comprising:
applying to the plant, a seed from which the plant is grown, or to an environment in which the plant is grown, a composition including chitin and a bacteria that secretes chitinase.

14. The process of claim 13, wherein the bacteria is Bacillus licheniformis (OBT 618).

15. The process of claim 13, wherein the bacteria is Paenibacillus ehimensis (PE).

16. The process of claim 13, wherein the bacteria is attached to the chitin with a binder.

17. The process of claim 13, wherein the bacteria and chitin are present in a liquid suspension.

18. The process of claim 13, wherein the chitin is present in the form of ground crab shell.

19. The process of claim 13, wherein the chitin is present in the form of ground shrimp shell.

20. A process for promoting plant growth, comprising:
applying to the plant, a seed from which the plant is grown, or to an environment in which the plant is grown, a composition that is a soluble portion of a fermented suspension of chitin and a bacteria that secretes chitinase.
